(19) United States
(12) Patent Application Publication
(10) Pub. No.: US 2015/0353886 A1
(43) Pub. Date: Dec. 10, 2015

(54) SALIVARY ESTRADIOL DETECTION METHODS AND USES FOR MONITORING OVULATION AND FERTILITY, AND SELECTION OR TIMING OF EGG RETRIEVAL AND/OR FERTILIZATION

(71) Applicants: SALIMETRICS, LLC, State College, PA (US); BOSTON IVF, INC., Waltham, MA (US)

(72) Inventors: Michael ALPER, Waltham, MA (US); Hans SCHROEDER, State College, PA (US); Jon PETERSON, State College, PA (US); Dionisios SAKKAS, Waltham, MA (US)

(21) Appl. No.: 14/763,627

(22) PCT Filed: Jan. 30, 2014
(86) PCT No.: PCT/US2014/013922

§ 371 Date: Jul. 27, 2015

Related U.S. Application Data
(60) Provisional application No. 61/758,457, filed on Jan. 30, 2013.

(51) Int. Cl. C12N 5/075; G01N 33/74; A61K 38/24

(52) U.S. Cl. CPC C12N 5/0609; A61K 38/24; G01N 33/743; G01N 2800/36

(57) ABSTRACT

This invention relates to methods to predict and monitor ovulation and fertility in female mammals by measuring the level of free salivary estradiol. Such information may be used to identify the appropriate timing for egg retrieval prior to in vitro fertilization (IVF), insemination procedures, natural or otherwise, and the timing for transfer of embryos. The ability to monitor salivary estradiol levels throughout the estrous cycle, with the optional application of an algorithm, will aid in identifying the peak time of fertility.

### RELATED APPLICATIONS

This application is the National Phase of International Application No. PCT/US2014/013922, filed Jan. 30, 2014, which designated the U.S. and that International Application was published under PCT Article 21(2) in English, which claims priority to U.S. Provisional Application No. 61/758,457, filed Jan. 30, 2013, all of which applications are expressly incorporated herein by reference in their entirety.

### FIELD OF THE INVENTION

The invention relates to, inter alia, identifying a peak or optimum time of fertility for egg retrieval or collection or for insemination (natural or artificial) by monitoring the levels of salivary estradiol. Such monitoring, or an algorithm aiding in the prediction thereof, is intended to enhance fertility and in turn the likelihood of pregnancy.

### INTRODUCTION

The female reproductive cycle begins with maturation and ovulation of the oocyte within the female reproductive tract. In normal sexual reproduction, fertilization of the oocyte by sperm also occurs in the reproductive tract. Assisted reproduction methodologies and technologies, such as in vitro fertilization (IVF), artificial insemination, egg production and subsequent storage or donation, use oocytes that are retrieved from the female at some point prior to fertilization and subsequently processed in vitro.

Estradiol levels have value to monitor ovulation and fertility in natural and controlled ovarian stimulation cycles by combining the assessment of increasing estradiol levels and increasing follicular size and number. Current methods of clinically assessing ovulation and fertility are through serum/plasma measurements of Estradiol (E2), Progesterone (P4), Leutinizing Hormone (LH), Follicle Stimulating Hormone (FSH), Anti-Mullerian Hormone (AMH), Inhibin, estrone-3-glucuronide (E3G) and other reproductive hormones. A regular series of tests are conducted throughout the estrous cycle or on specific days to identify the event of ovulation, to time embryo transfer after in vitro fertilization or to generally assess the fertility status of a patient.

Knowledge of reproductive hormones is a good gauge of optimal fertility and extremely useful for In Vitro Fertilization (IVF) and Intra Uterine Insemination (IUI) procedures. In addition to natural ovulation, estradiol levels are crucial in monitoring follicular development in controlled ovarian hyperstimulation (COH) cycles during IVF. Estradiol levels increase significantly, especially in the case of COH where the patient generally undergoes the harvesting and release of more than one oocyte (Delaney A, Jensen J R and D Marbeck. Fertility Testing. Clin Lab News. November 2012 38(11))

Less obvious and in some cases, fertility problems with some women have been linked to stress (A Negro-Vilar, Environ Health Perspect. 1993 July; 101(Suppl 2): 59-64). Saliva testing is non-invasive, whereas collection of serum/plasma is accomplished through venipuncture with a needle connected to a vacutainer which requires a trained professional. Such invasive procedures can be stressful (Balcombe, J P, Barnard, N D and C Sandusky. Journal of the American Association for Laboratory Animal Science, Volume 43, Number 6, November 2004, pp. 42-51(10)).

### SUMMARY

Estradiol levels can be used as an adjunct to follicular size and number to determine the readiness of female subjects for insemination (artificial or natural) or the retrieval or collection of eggs, optionally in conjunction with analysis or evaluation of one or more other predetermined clinical parameters indicative of readiness for egg retrieval, collection or insemination of the female patient. Accordingly, in one embodiment, the invention is directed to methods and uses for assisted reproduction methodology/technology to assess a patient's readiness to undergo in vitro fertilization (IVF) procedures and/or insemination at an optimal time.

Saliva testing is non-invasive and as disclosed herein estradiol levels in saliva correlate to serum and/or plasma levels. Salivary collection allows a patient to prepare a sample in the comfort of the home environment to bring into the clinic for out-patient procedures.

In practice, a female subject's estradiol level can be monitored at regular or directed time points and/or intervals throughout her estrous cycle in parallel with ultrasound measurements to determine follicular size and number. In addition, estradiol levels in the femal subject can indicate hyper ovulation syndrome a potentially life threatening condition. The development of mathematical algorithms can be used to predict optimum times of ovulation and fertility during an in vitro fertilization treatment or insemination.

In accordance with the invention, there are provided methods and uses for stimulating production of eggs in a female mammal for in vitro fertilization. In one embodiment, a method or use includes administering a hormone therapy that stimulates egg production in a female mammal, obtaining or providing a saliva sample from the female mammal, measuring the amount of estradiol in the saliva sample, and comparing the amount of saliva estradiol to a previously determined amount of estradiol from the female mammal in order to identify an increase in saliva estradiol. An estradiol peak reflects increased egg production in the female mammal, thereby stimulating egg production in the female mammal for in vitro fertilization.

In accordance with the invention, there are also provided methods and uses for selecting a female for egg production or retrieval. In one embodiment, a method or use includes, administering a hormone therapy that stimulates egg production in a female mammal, obtaining or providing a saliva sample from the female mammal, measuring the amount of estradiol in the saliva sample, comparing the amount of saliva estradiol to a previously determined amount of saliva estradiol from the female mammal in order to identify an increase in saliva estradiol, wherein an estradiol peak reflects increased egg production in the female mammal; and selecting the female for egg production or retrieval, and optionally retrieving said eggs.

In accordance with the invention, there are additionally provided methods and uses for providing eggs for in vitro fertilization or increasing probability of pregnancy in a female mammal. In one embodiment, a method or use includes administering a hormone therapy that stimulates egg production in a female mammal, obtaining or providing a saliva sample from the female mammal, measuring the amount of estradiol in the saliva sample, and comparing the amount of saliva estradiol to a previously determined amount of saliva estradiol from the female mammal in order to identify an increase in estradiol. An estradiol peak reflects increased egg production in the female mammal, and then retrieving eggs from the female for subsequent in vitro fertilization, or performing in-utero insemination on the female mammal.

In accordance with the invention, there are further provided methods and uses for determining a peak of estradiol in a female mammal. In one embodiment, a method or use includes obtaining or providing a saliva sample from a female mammal having received a hormone therapy that stimulates egg production, measuring amounts of estradiol in the saliva; and repeating the steps a plurality of times in order to determine estradiol in saliva over a period of time and a peak of estradiol in the female mammal.

In accordance with the invention, there are moreover provided methods and uses for determining increased fertility in a female mammal. In one embodiment, a method or use includes obtaining or providing a saliva sample from a female mammal, optionally said female having received a hormone therapy that stimulates egg production, measuring amounts of estradiol in the saliva; and repeating the steps a plurality of times in order to determine estradiol in saliva over a period of time and a peak of estradiol in the female mammal, wherein the peak of estradiol indicates increased fertility in a female mammal.

In accordance with the invention, there are still also provided methods and uses for stimulating production of eggs in a female mammal for in vitro fertilization. In one embodiment, a method or use includes administering a hormone therapy that stimulates egg production in a female mammal, obtaining or providing a saliva sample from the female mammal, measuring amounts of estradiol in the saliva sample, converting the amount of estradiol measured in the saliva to a serum equivalent unit (SEU) of estradiol, and comparing the SEU of estradiol to a previously determined SEU of estradiol for the female mammal in order to identify an increase in estradiol. An estradiol peak reflects increased egg production in the female mammal, thereby stimulating egg production in the female mammal for in vitro fertilization.

In accordance with the invention, there are still additionally provided methods and uses for selecting a female for egg production or retrieval. In one embodiment, a method or use includes administering a hormone therapy that stimulates egg production in a female mammal, obtaining or providing a saliva sample from the female mammal, measuring amounts of estradiol in the saliva sample, converting the amount of estradiol measured in the saliva to a serum equivalent unit (SEU) of estradiol, and comparing the SEU of estradiol to a previously determined SEU of estradiol for the female mammal in order to identify an increase in estradiol. An estradiol peak reflects increased egg production in the female mammal, and then selecting the female for egg production or retrieval.

In accordance with the invention, there are still further provided methods and uses for providing eggs for in vitro fertilization or increasing probability of pregnancy in a female mammal. In one embodiment, a method or use includes administering a hormone therapy that stimulates egg production in a female mammal, obtaining or providing a saliva sample from the female mammal, measuring amounts of estradiol in the saliva sample, converting the amount of estradiol measured in the saliva to a serum equivalent unit (SEU) of estradiol, and comparing the SEU of estradiol to a previously determined SEU of estradiol for the female mammal in order to identify an increase in estradiol. An estradiol peak reflects increased egg production in the female mammal, and then retrieving or collecting eggs from the female for subsequent in vitro fertilization, or then performing in-utero insemination on the female mammal.

In accordance with the invention, there are still moreover provided methods and uses for determining a serum equivalent unit (SEU) of estradiol in a female mammal. In one embodiment, a method or use includes obtaining or providing a saliva sample from a female mammal having received a hormone therapy that stimulates egg production, measuring amounts of estradiol in the saliva sample, and converting the amount of estradiol measured in the saliva to a SEU of estradiol.

In accordance with the invention, there are yet also provided methods and uses for determining a peak of estradiol in a female mammal. In one embodiment, a method or use includes obtaining or providing a saliva sample from a female mammal having received a hormone therapy that stimulates egg production, measuring amounts of estradiol in the saliva sample, converting the amount of estradiol measured in the saliva to a serum equivalent unit (SEU) of estradiol; and repeating the steps a plurality of times in order to determine SEU of estradiol over a period of time and thereby a peak of estradiol in the female mammal.

In various aspects of invention methods and uses, steps of the methods are repeated 2 or more times (i.e., a plurality of times, such as 2, 3, 4, 5, 6, 7, 8, 9, 10, 11, 12, 13, 14, 15, 16, 17, 18, 19, 20, 21, 22, 23, 24, 25, or more times). In a particular non-limiting aspects, steps of hormone administration, obtaining or providing a sample of saliva, measuring the amount of estradiol in the sample, determining the SEU of estradiol, comparing the estradiol amount or SEU sample to a previously determined or baseline level of estradiol or SEU, etc., are performed a plurality of times. For example, in any invention embodiment, steps of measuring and comparing can be performed a plurality of times in order to determine amounts of estradiol in the female mammal over a period of time, and/or in order to determine a peak of estradiol in a female mammal.

In additional various aspects of all invention methods and uses, follicle size or follicle number in the female mammal is determined. In additional various aspects of invention methods and uses the female mammal is selected, the eggs retrieved or collected, or the in-utero insemination is performed based upon a combination of any of the amount or peak of saliva estradiol, follicle size and follicle number in the female mammal. In further various aspects of invention methods and uses, the female mammal is selected, the eggs retrieved or the in-utero insemination is performed based upon a combination of any of SEU of estradiol, follicle size and follicle number in the female mammal.

In still also various aspects of all invention methods and uses, the estradiol amount or SEU is compared to a baseline level of estradiol or SEU determined in the female mammal prior to the female mammal receiving the hormone therapy that stimulates egg production. A baseline level of estradiol can be determined in the female mammal during the estrous cycle prior to the female mammal receiving the hormone therapy that stimulates egg production.

In still additional various aspects of all invention methods and uses, the estradiol amount or SEU is compared to a level of estradiol or SEU determined in the female mammal after the female mammal began receiving the hormone therapy that stimulates egg production.

In still further various aspects of all invention methods and uses, the saliva sample is obtained or provided on or from the same day, or within 1-21 days or within 1-14 days before or after the female mammal began receiving the hormone therapy that stimulates egg production.

In still more various aspects of all invention methods and uses, a plurality of saliva samples are obtained or provided on or from before or after the female mammal began receiving the hormone therapy that stimulates egg production, and amounts of estradiol measured in the saliva; or a plurality of saliva samples are obtained or provided on or from before or after the female mammal began receiving the hormone therapy that stimulates egg production, and amounts of estradiol measured in the saliva and converted to a serum equivalent unit (SEU) of estradiol.

In yet also various aspects of all invention methods and uses, estradiol in saliva is measured once per day for a period of 1-21 days or once per day for a period of 1-14 days before or after the female mammal began receiving the hormone therapy that stimulates egg production.

In yet additional various aspects of all invention methods and uses, the estradiol in saliva is measured one or more times 1-30 days before the female mammal began receiving the hormone therapy that stimulates egg production, and/or one or more times 1-21 after the female mammal began receiving the hormone therapy that stimulates egg production.

In more particular aspects of all invention methods and uses, estradiol in saliva is measured with a detectable assay, such as an antibody immunoassay. Particular assay include measurement by way of an enzyme immunoassay (EIA) or enzyme linked immunosorbent assay (ELISA).

In yet further various aspects of all invention methods and uses, a female mammal has received a plurality of hormone administrations. Such hormones include, follicle stimulating hormone (FSH).

Various additional non-limiting clinical parameters measured, analyzed or evaluated in accordance with all invention methods and uses include a peak of estradiol greater than about 25 pg/mL estradiol in saliva, or an SEU of estradiol within a range of between about 1,000 and 4,000 pg/mL, or further comprising determining follicle size or the number of follicles in the female mammal. In particular non-limiting aspects, wherein at least 1 or 2 follicles 18 mm or greater in size, or wherein half or more of all the follicles are at least 15 mm in size, or wherein the number of follicles is at least 8, which indicates increased egg production.

In yet more various aspects of all invention methods and uses, hormone therapy is discontinued if the female mammal if saliva estradiol is greater than a threshold amount, for example, greater than about 40 pg/mL, or if the SEU of estradiol is greater than about 4,000 pg/mL.

In additional optional steps in accordance with all invention methods and uses, eggs are retrieved or collected from the female mammal. In a particular aspect, 5-20 eggs are retrieved or collected from the female mammal.

In further optional steps in accordance with all invention methods and uses, eggs from the female mammal are subsequently fertilized, stored or donated to a recipient. In particular aspects, in vitro fertilization on one or more of the retrieved or collected from the female mammal is performed, or in-utero insemination (IUI) is performed on the female mammal.

In accordance with all invention methods and uses, the amount of saliva estradiol is converted to a SEU of estradiol using a mathematical algorithm. In particular non-limiting embodiments, the amount of saliva estradiol is converted to the SEU of estradiol using a mathematical algorithm set forth as:

SEU=(0.027*(SalE2⁴))−(2.2*(SalE2³))+(54.41*(SalE2²))−(299.1*SalE2)+585.44,

where SalE2 is saliva estradiol amount. In additional alternative non-limiting embodiments, the amount of saliva estradiol is converted to a SEU of estradiol using a mathematical algorithm set forth as:

SEU=−2.5x³+73.3x²−401x+806;

SEU=−2.1x²+258x−766; or

SEU=85.3e^{0.24x},

where x=saliva estradiol amount.

### DESCRIPTION OF DRAWINGS

FIG. 1 shows data indicating that salivary estradiol measurements were found to have a correlation to those made with serum plasma, with a R2=0.661.

FIG. 2 shows development of serum equivalent units (SEU) model, with inputs of saliva, serum and/or follicle size and number determination.

FIG. 2A shows number of samples for model development.

FIG. 2B shows rolling average of each 20 Saliva estradiol values according to day of cycle, from 25 patients. X-axis=day of cycle; Y-axis=estradiol amounts (pg/ml).

FIG. 2C shows rolling average of each 20 Serum estradiol values according to day of cycle, from 25 patients. X-axis=day of cycle; Y-axis=estradiol amounts (pg/ml).

FIG. 2D shows an exemplary 4^{th} degree polynomial fit for data based upon Serum and Saliva estradiol levels, from 25 patients.

FIG. 2E shows exemplary implementation of SEU, by way of a flow diagram.

FIG. 3 shows data from 4 patients comparing serum E2 values (Blue) with Saliva E2 values (Green) and Serum Equivalent Units (SEU) (Red) calculated using a polynomial algorithm.

FIGS. 4A-4F show follicle size and number in pooled millimeter (mm) plotted against salivary estradiol values in pg/mL for 6 patients (patients #1-#6, data also shown in Table 1).

### DETAILED DESCRIPTION

The invention relates to methods and uses for stimulating production of eggs, or selecting a female for egg production or retrieval or insemination, or providing eggs for subsequent IVF or IUI, storage or donation from a female mammal. The invention also relates to assessing the likelihood of conception (or, successful implantation) of a female mammal. The invention further relates to methods and uses in diagnosis in order to determine timing of egg retrieval or the suitability of a female mammal for egg retrieval, and or subsequent IUI or IVF treatment. The invention moreover relates to methods and uses in determining increased fertility in a female mammal and a peak of estradiol in a female mammal.

Methods and uses of the invention are directed to, among other things, fertility treatments and therapies, enhancing and promoting fertility treatments and therapies, monitoring and scheduling fertility treatments and therapies, stimulating and promoting egg production, timing of egg retrieval and optional subsequent fertilization, storage or donation, selection of female mammals for egg retrieval and optional subsequent fertilization, storage or donation.

The term “fertility treatment” or “therapy” refers to the use of medications to induce ovulation or to stimulate ovaries to produce more than one mature egg, for example, in controlled ovarian hyperstimulation (COH). A mature egg is one that can be fertilized to develop into an embryo.

“Fertility treatments” and “therapies” are also intended to include conception that is aided by artificial insemination (i.e., intra-uterine insemination or JUT), in-vitro fertilization or IVF, and/or intra-cytoplasmic sperm injection or ICSI. The term “fertility treatment” or “therapy” also includes the enhancement of natural, induced, or stimulated cycle by cycle monitoring, with or without medical intervention or medications; in this respect, the aim of the fertility treatments is to improve the viability of the embryos that are conceived in vivo (e.g., after JUT or by natural conception) or that are transferred to the uterus, after IVF, ICSI, embryo cryopreservation-thawing, or any other assisted reproductive methodology. Additional examples of fertility treatments and therapies include Gamete Intrafallopian Transfer Procedure (GIFT), Zygote Intrafallopian Transfer Procedure (ZIFT) and Therapeutic Donor Insemination (TDI).

The term “assisted reproductive methodology” or “assisted reproductive technology” and grammatical variations thereof refer to treatments and therapies that involve in vitro (outside of the body) manipulation of gametes (eggs and sperm at any stage of maturity). Exemplary types include without limitation: (i) in vitro fertilization (IVF), which entails surgical retrieval of eggs from the body and fertilization in vitro followed by in vitro culture of the embryos and transfer of the embryos back into the uterus; (ii) intra-cytoplasmic sperm injection (ICSI), which is the same as IVF except that sperm is singly microinjected into each egg to bypass the natural process of fertilization; (iii) gamete intra-fallopian transfer (GIFT), where retrieved eggs and processed sperm are placed into fallopian tubes in a surgical procedure (laparoscopy) and subsequent fertilization and conception occurs within the body; (iv) use of cryopreserved embryos from IVF or ICSI procedures; (v) use of eggs from a female egg donor; (vi) use of sperm from a male donor; (vii) placement of embryos into the womb of a surrogate mother; and (viii) use of cryopreserved eggs (not embryos) from a female or sperm from a male.

The invention provides a method for improving fertility, implantation and/or pregnancy rates in a female mammal. The term “improving” as used herein in the context of an effect on fertility, implantation or pregnancy includes any measurable improvement or increase in frequency of occurrence of egg production, implantation or pregnancy in a female mammal for example when compared with the level or frequency of egg production, implantation or pregnancy in one or more non-treated females or when compared to the level or frequency of egg production, or occurrence of implantation or pregnancy in the same female observed at an earlier time point.

An “egg” or “oocyte” refers to a mature animal ovum, which is the final product of oogenesis, as well as precursor forms being the oogonium, the primary oocyte and the secondary oocyte, respectively. Egg developmental parameters include without limitation, morphology, number, developmental stage, developmental grade, molecular markers of development, and combinations thereof. The retrieval or collection of eggs from a female mammal involves aspiration of cumulus oocyte complexes (COCs) from the follicles of the female. Typically, a COC is subsequently matured in vitro. Accordingly, retrieving or collecting eggs from the follicles of female mammals prior to in vitro maturation of the oocytes should therefore be understood to include cumulus oocyte complexes.

Ovulation in a female can be defined to have occurred when the majority of follicles surrounding an oocyte have disappeared. Methods and uses in accordance with the invention can therefore optionally include, but are not limited to, the use of devices, techniques and methodologies that detect timing of ovulation, such as transcutaneous ultrasonography. Monitoring of ovulation timing permits retrieval of eggs prior to ovulation, as a result of COH and other ovulation management therapies. Monitoring the time of ovulation can be valuable for egg retrieval and collection in the methods and uses as set forth herein.

Methods and use of the invention including determining, measuring or analyzing estradiol from a female mammal, such as saliva present in the female or obtained from the female. As used herein, the terms “determining,” “measuring” or “analyzing” in the context of quantifying estradiol amounts are used interchangeably herein and can refer to absolute or to relative quantification. Measuring involves manipulation or processing such as a laboratory procedure involving one or more of obtaining, isolating, processing, contacting, purifying, manipulating, or determining steps practiced with a sample or specimen, such as saliva, the amount of estradiol which is distinct from any mental step. Absolute quantification may be accomplished by inclusion of a known control amount of estradiol and analyzing the control estradiol amount and the amount in a sample such as saliva. Alternatively, relative quantification can be accomplished by comparing signals between a control and one or more samples to quantify the estradiol amount in a sample. When the terms are used in reference to measurement or detection, any means of assessing the relative amount, including the various methods set forth herein and known in the art, performed by the hand of man, is contemplated.

As set forth herein, the methods and uses of the invention can be performed in solution, in solid phase, in silica, in vitro, ex vivo, and in vivo. In one embodiment, a method includes contacting a sample (saliva) present in a female mammal, or obtained from a female mammal, with a detection reagent that detects estradiol, and measuring the amount of estradiol in the sample present in or obtained from the female mammal. Based upon the determination of the amount of estradiol a course of action such as a procedure as set forth herein (hormone modulation or administration to the female mammal, or egg retrieval or collection from the female mammal, insemination of the female mammal, IVF of the female mammal, or storage or donation to a recipient, etc.) can be subsequently taken.

In one embodiment, a plurality of estradiol measurements is determined over a period of time in a female mammal. The time interval between measurements can be within minutes (e.g., 1-60), hours (e.g., 1-24), days (e.g., 1-30) or months (e.g., 1-3), and used to determine a peak of estradiol. Such measurements over a period of time are useful, for example, to determine a peak of estradiol. A peak is determined by comparing 2 or more values of estradiol to each other, such as saliva amounts or SEU of estradiol, and can be carried out by visual inspection, or by using a machine or apparatus capable of detection of a signal.

As used herein, a “peak” of estradiol refers to estradiol amounts that have increased, by comparison to a previously determined amount of estradiol or a baseline estradiol level in a female mammal. The peak of estradiol can be used as an indicator of increased egg production and increased number and/or size of follicles that a clinician considers to be an appropriate indicator for timing of egg retrieval or IUI, and optionally subsequent artificial fertilization such as IVF, or storage, or donation to a recipient. A peak of estradiol generally falls within a broad range, for example, in saliva a range of 20-60 pg/ml, and in serum a range from 1000-3600 pg/ml, but is variable in female mammals and is therefore a relative value typically established within a given female. The estradiol peak is therefore established by way of comparison to a prior estradiol determination or a baseline estradiol level in the female, for example, established during the normal estrus cycle absent COH. Optionally the comparison is to a prior determined amount of estradiol in the female such that there is a measurable increase of estradiol. Accordingly, a peak of estradiol is not an absolute value but reflects a relative increase in a given female mammal.

Furthermore, a peak of estradiol need not be the highest attainable amount of estradiol in a given female mammal over a given period of time. A peak is considered to be reached by the clinician if it coincides with the criteria of increased egg production and increased number and/or size of follicles that a clinician considers to be appropriate for timing of egg retrieval, and subsequent manipulation, storage, donation, etc. Examples of peak saliva estradiol levels and associated follicle size are shown in Example 3, occurring on days 9 to 11 of stimulation and an estradiol range between about 20-60 pg/ml.

As disclosed herein, in invention methods and uses, a sample can be present in or obtained from a female mammal, and a plurality of samples can be present in or obtained from a given female subject over the course of a period of time, such as one or more minutes (1-60 minutes), hours (1-24 hours) days (1-30 days) or months. Thus, invention methods and uses include analysis of estradiol in samples present in or obtained from a given female subject once every 1-60 minutes, 1-24 hours, or over the course of as few as 1 but as many as 30 days. Accordingly, estradiol measurement can be determined in samples present in or obtained from a female mammal over a period of time, including but not limited to every 1-60 minutes, 1-24 hours or 1-30 days.

The single or plurality of samples can be measured for estradiol amounts close in time, for example, within 1-60 minutes or 1-24 hours of the sample being taken, or later (e.g., within 1-3 days when the sample was obtained). The timing of estradiol measurement in relation to the obtaining a sample will depend upon the objective or purpose of estradiol measurement.

A sample includes a biological sample. Biological samples include any sample having biological material. Biological samples include saliva, serum, plasma and blood. A biological sample is desirably suitable for measuring or analyzing estradiol amounts.

A biological sample can be transformed, processed or manipulated, for example, to determine the presence of, or measure or analyze, estradiol amounts or levels. Typically, a biological sample is transformed or processed in order to measure or analyze amounts of estradiol. Thus, samples also include material purified, isolated, derived from, extracted from, or obtained from a biological sample such as saliva, serum, plasma and blood.

During the course of estradiol analysis, for example, over a period of time, a female mammal may undergo various treatments or therapies. For example, a female mammal may undergo a treatment or therapy administered singly or as a combination, or separately, concurrently or in sequence (sequentially) in accordance with the methods and uses described herein. Such treatments and therapies include a single or multiple dose e.g., one or more times hourly, daily, weekly, or monthly, or for as long as appropriate.

Accordingly, methods and uses of the invention include an optional treatment or administration of a therapy to a subject. Such treatments and therapies in the context of the invention include those for increasing fertility, modulating ovulation and maturation of eggs, and/or production of eggs for retrieval and subsequent insemination, in vitro fertilization, storage or donation to a recipient. Particular non-limiting examples include delivery of hormones. Hormone manipulation typically will include treatments that modulate one or more reproductive tissues, such as ovaries, uterus or pituitary gland.

Non-limiting examples of hormones include without limitation, sex and pregnancy hormones, such as follicle stimulating hormone (FSH) which induces follicle growth and egg production. Additional hormones that typically may be used include estradiol, progesterone, gonadotropin releasing hormone agonists, gonadotropin releasing hormone antagonists, gonadotropins, and synthetic agonists which cause the release of the female's hormones. Such hormones include those that suppress the female's own release of hormones so that other hormones may be administered and greater control of the egg production cycle is achieved. Further therapies include clomiphene citrate, which stimulates the pituitary gland to release hormones, and human chorionic gonadotropins (hCG) which promotes final maturation of oocytes in the follicles. Administration of hCG is timed according to the size of the leading follicles as measured by ultrasound scanning as well as the levels of estrogen in the blood. Oocyte recovery is typically 34 to 36 hours after the administration of the hCG. Progesterone can be administered after ovulation or after fertilization to produce an endometrium which is situated for fertilized egg implantation.

Administration of hormones may be undertaken by any generally suitable procedure, including intravenous injection, intramuscular injection, intraperitoneal injection, subcutaneous injection, or by other routes including oral, topical, vaginal, or rectal. Dosage forms may be administered as controlled release formulations where appropriate.

Such administration can be prior to, substantially contemporaneously with or after a subject has been evaluated for estradiol amounts, or a sample has been obtained from the subject that is to be evaluated for saliva estradiol amount. Such administrations can be multiple administrations of a therapy, prior to, substantially contemporaneously with or after a subject has been evaluated for estradiol amounts, or a sample has been obtained from the subject that is to be evaluated for saliva estradiol amount.

A “subject” refers to a female mammal e.g., a human female. The invention methods and uses are applicable to any female mammal, including non-human females. The term “female mammal” therefore includes animals, such as humans, non-human primates (apes, gibbons, gorillas, chimpanzees, orangutans, macaques), domestic animals (dogs and cats), a farm animal (poultry such as chickens and ducks, horses, cows, goats, sheep, pigs), and experimental animals (mouse, rat, rabbit, guinea pig). Invention methods and uses are also applicable, for example, to captive breeding programs of endangered species or commercial breeding of livestock such as cattle, swine or horses.

Subjects include those having reduced or decreased fertility, or those at risk of reduced or decreased fertility, as well as subjects that wish to undergo fertility treatment or improve or promote fertility by way of any acceptable method. Subjects therefore include candidates for natural or artificial insemination, such as intra-uterine insemination and in vitro fertilization, as well as subjects who wish to produce eggs for subsequent storage or for donation to a recipient.

The invention methods and uses are also applicable to detecting high, supraphysiologic levels of estrogen, attained during COH, which can result in an adverse effect on the outcome of fertility treatment, for example, Ovarian Hyper Stimulation (OHSS). Another example is when significant decreases in pregnancy and implantation rates have been observed when estradiol concentrations were >10,000 pmol/L compared with patients having lower estradiol concentrations. High serum estradiol concentrations, in addition to other clinical parameters (e.g., blood cell count, hematocrit, bloating, abdominal discomfort, difficulty walking, etc.) on the day of HCG injection in IVF patients, regardless of the number of oocytes retrieved, have been reported to be detrimental to uterine receptivity. A significant reduction in implantation and pregnancy rates occurred in almost all women with a serum estradiol concentration greater than 20,000 pmol/L. Excessive estradiol production during COH may lead to insufficient secretory transformation of the endometrium and discordant glandular and stromal development at a time that coincides with the period of maximum uterine receptivity. In addition, there are possible adverse effects on the embryo that could reduce the chance for implantation. Thus, the invention methods and uses are applicable to identifying female mammals whose estradiol levels are undesirably high, in which case such females can be withdrawn from COH.

As disclosed herein, estradiol can be measured and/or analyzed by using a detection reagent. Detection reagents include molecules that bind to estradiol. One example is an antibody in an immunoassay to detect, measure or analyzed estradiol. Accordingly, antibodies and subsequences thereof that bind to estradiol can be used to detect estradiol and according to the invention measure estradiol amounts.

Other methods of measuring estradiol are known to those skilled in the art including lateral flow, magnetic bead or other point of care assays. Non-limiting exemplary detection, measurement and analysis methods include Western blot, immunoblot, enzyme-linked immunosorbant assay (ELISA), radioimmunoassay (RIA), immunoprecipitation, surface plasmon resonance, chemiluminescence, absorption, emission, fluorescent polarization, phosphorescence, or matrix-assisted laser desorption/ionization time-of-flight (MALDI-TOF) mass spectrometry. Amounts of estradiol also can be measured using a functional assay, based upon a function of the hormone, such as receptor binding, signal transduction, etc.

The term “antibody” refers to a protein that binds to other molecules (antigens) via heavy and/or light chain variable domains, V_{H} and/or V_{L}, respectively. An “antibody” refers to any monoclonal or polyclonal immunoglobulin molecule, such as IgG, IgA, IgD, IgE, IgM, and any subclass thereof (e.g., IgG₁, IgG₂, IgG₃ or IgG₄). Antibodies include full-length antibodies that include two heavy and two light chain sequences. Antibodies can have kappa or lambda light chain sequences, either full length as in naturally occurring antibodies, mixtures thereof (i.e., fusions of kappa and lambda chain sequences), and subsequences/fragments thereof. Naturally occurring antibody molecules contain two kappa or two lambda light chains.

Antibodies and subsequences thereof include mammalian, primatized, humanized and fully human antibodies and subsequences thereof. Antibodies and subsequences thereof include those produced or expressed by or on transformed cells or hybridomas, or B cells, or those produced synthetically or by other organisms (plant, insect, bacteria, etc.).

Antibodies include polyclonal and monoclonal antibodies. A “monoclonal” antibody refers to an antibody that is based upon, obtained from or derived from a single clone, including any eukaryotic, prokaryotic, or phage clone. A “monoclonal” antibody is therefore defined structurally, and not the method by which it is produced.

Antibodies include subsequences. Non-limiting representative antibody subsequences include but are not limited to Fab, Fab′, F(ab′)₂, Fv, Fd, single-chain Fv (scFv), disulfide-linked Fvs (sdFv), V_{L}, V_{H}, Camel Ig, V-NAR, VHH, trispecific (Fab₃), bispecific (Fab₂), diabody ((V_{L}−V_{H})₂ or (V_{H}-V_{L}), triabody (trivalent), tetrabody (tetravalent), minibody ((scFᵥ-C_{H}3)₂), bispecific single-chain Fv (Bis-scFv), IgGdeltaCH2, scFv-Fc, (scFv)₂-Fc, affibody, aptamer, avimer or nanobody, or other antigen binding subsequences of an intact immunoglobulin. Antibodies include those that bind to more than one epitope (e.g., bi-specific antibodies), or antibodies that can bind to one or more different antigens (e.g., bi- or multi-specific antibodies).

Antibodies and subsequences thereof can be produced or are available commercially or from other sources. For example, antibodies that bind to estradiol can be produced using standard immunological methods known to one skilled in the art.

A mammalian antibody is an antibody produced by a mammal, transgenic or non-transgenic, or a non-mammalian organism engineered to produce a mammalian antibody, such as a non-mammalian cell (bacteria, yeast, insect cell), animal or plant. A “human” antibody means that the amino acid sequence of the antibody is fully human, i.e., human heavy and human light chain variable and human constant regions. A “humanized” antibody, means that the amino acid sequence of the antibody has non-human amino acid residues (e.g., mouse, rat, goat, rabbit, etc.) of one or more complementarity determining regions (CDRs) that specifically bind to the desired antigen in an acceptor human immunoglobulin molecule, and one or more human amino acid residues in the Fv framework region (FR), which are amino acid residues that flank the CDRs.

A detection reagent such as an antibody can be labeled or tagged in order to be detectable. Detectable labels, markers and tags include labels suitable for measurement, analysis and/or quantitation, and include any composition detectable by enzymatic, biochemical, spectroscopic, photochemical, immunochemical, isotopic, electrical, optical, chemical or other means. A detectable label can be attached (e.g., linked conjugated) to the detection reagent, or be within or be one or more atoms that comprise the detection reagent. As the structure of detection reagents can include one or more of carbon, hydrogen, nitrogen, oxygen, sulfur, phosphorous, etc., radioisotopes of any of carbon, hydrogen, nitrogen, oxygen, sulfur, phosphorous, etc., can be included within a labeled detection reagent.

Non-limiting exemplary detectable labels also include a radioactive material, such as a radioisotope, a metal or a metal oxide. Radioisotopes include radionuclides emitting alpha, beta or gamma radiation. In particular embodiments, a radioisotope can be one or more of: C, N, O, H, S, Cu, Fe, Ga, Ti, Sr, Y, Tc, In, Pm, Gd, Sm, Ho, Lu, Re, At, Bi or Ac. In additional embodiments, a radioisotope can be one or more of: ³H, ¹¹C, ¹⁴C, ¹³N, ¹⁸O, ¹⁵O, ³²P, ³³P, ³⁵S, ¹²⁵I, or ¹³¹I.

Further non-limiting exemplary detectable labels include contrast agents (e.g., gadolinium; manganese; barium sulfate; an iodinated or noniodinated agent; an ionic agent or nonionic agent); magnetic and paramagnetic agents (e.g., iron-oxide chelate); nanoparticles; an enzyme (horseradish peroxidase, alkaline phosphatase, β-galactosidase, or acetylcholinesterase); a prosthetic group (e.g., streptavidin/biotin and avidin/biotin); colorimetric labels such as colloidal gold or colored glass or plastic (e.g., polystyrene, polypropylene, latex, etc.) beads; a fluorescent material or dye (e.g., umbelliferone, fluorescein, fluorescein isothiocyanate, rhodamine, dichlorotriazinylamine fluorescein, dansyl chloride, texas red, rhodamine); a luminescent material (e.g., luminol); or a bioluminescent material (e.g., green fluorescent protein, luciferase, luciferin, aequorin). A label can be any imaging agent that can be employed for detection, measurement, analysis and/or quantitation (e.g., for computed axial tomography (CAT or CT), fluoroscopy, single photon emission computed tomography (SPECT) imaging, optical imaging, positron emission tomography (PET), magnetic resonance imaging (MRI), gamma imaging).

A detectable label can also be linked or conjugated (e.g., covalently) to an detection reagent. In various embodiments a detectable label, such as a radionuclide or metal or metal oxide can be bound or conjugated to the detection reagent, either directly or indirectly. A linker or an intermediary functional group can be used to link a detection reagent to a detectable label.

The term “contact” and grammatical variations thereof means conditions allowing a physical interaction (direct or indirect) between two or more entitites (e.g., a detection reagent and estradiol). In one example, contact means interaction (e.g., binding) of a detection reagent and a biological sample, such as saliva, serum, plasma, etc.

For methods and uses for detection, measurement or analysis of estradiol amounts used herein includes in solution, in solid phase, in situ, in vitro, ex vivo, in vivo. Thus, methods and uses of the invention include contact under conditions allowing the detection reagent to bind to estradiol indicative of estradiol amounts or levels.

The term “bind,” or “binding,” when used in reference to an detection reagent such as an antibody means that the binding moiety interacts at the molecular level with all or a part of estradiol. Typically binding is sufficiently specific or selective for a target, i.e., is statistically significantly higher than the background or control binding for the assay. The term “specifically binds” refers to the ability to preferentially or selectively bind to a target, for example, a detection reagent that binds to estradiol. Specific and selective binding can be distinguished from non-specific/selective binding in the assays disclosed herein and others known to the skilled artisan. For example, when performing an immunoassay, controls typically include a reaction well/tube that contains an antibody or antigen binding fragment alone (i.e., in the absence of protein sample), wherein an amount of reactivity (e.g., non-specific binding to the well) by the antibody or antigen binding fragment thereof in the absence of protein sample is considered to be background.

A detection reagent can be either in a free state, in solution or in solid phase, such as immobilized on a substrate or a support (e.g., solid). Examples of substrates and supports include a multiwall (e.g., microtiter) plate, a bead or sphere, a tube or vial, a microarray or any other suitable substrate or support. Immobilization can be by passive adsorption (non-covalent binding) or covalent binding between the substrate or support and the detection reagent, or indirectly by attaching the detection reagent to another reagent which other reagent is in turn attached to the substrate or support (e.g., a ligand-receptor system, for example, where a molecule is grafted onto the detection reagent and the corresponding receptor immobilized on the substrate or support, as exemplified by the biotin-streptavidin system).

Substrates and supports include dry solid medium (e.g., cellulose, polyester, nylon, or mixtures thereof etc.), such as glass, silica, plastic, polyethylene, polystyrene, polypropylene, polyacetate, polycarbonate, polyamide, polyester, polyurethane, or polyvinylchloride. One such material, an organic polymer such as polypropylene, is chemically inert and hydrophobic, and has good chemical resistance to a variety of organic acids, organic agents, bases, salts, oxidizing agents, and mineral acids. Additional non-limiting examples include polyethylene, polybutylene, polyisobutylene, polybutadiene, polyisoprene, polyvinylpyrrolidine, polytetrafluroethylene, polyvinylidene difluoride, polyfluoroethylene-propylene, polyethylenevinyl alcohol, polymethylpentene, polycholorotrifluoroethylene, polysulfonones, hydroxylated biaxially oriented polypropylene, aminated biaxially oriented polypropylene, thiolated biaxially oriented polypropylene, etyleneacrylic acid, thylene methacrylic acid, nylons, and blends or copolymers thereof.

Substrates and supports include structures having sections, compartments, wells, containers, vessels or tubes, separated from each other to avoid or prevent cross-contamination or mixing with each other or with other reagents. Multi-well plates, which typically contain 6, 12, 26, 48, 96, to 1000 wells, are one particular non-limiting example of such a structure. Substrates also include two- or three-dimensional arrays.

Estradiol levels can be measured and the raw estradiol data and/or the comparison data generated can optionally be stored and/or managed via paper charts or files, in electronic form (e.g., a computer readable medium, server or database) in a personal health record, or a combination of any of the foregoing. The data generated will typically be prepared by the healthcare provider. For conversion of saliva estradiol amounts to SEU of estradiol, the salivary estradiol data can optionally be processed by a computer to obtain the SEU of estradiol using a mathematical algorithm, for example, as set forth herein. In one embodiment, data will be provided in a report including a description of the analysis, interpretation of the data, and optionally in a format to facilitate communication between the clinician and the patient.

Medical professionals are accustomed to measuring estradiol using serum tests. In order to improve acceptability among clinicians, an algorithm has been developed to convert salivary estradiol results into serum equivalent units (SEU) of estradiol. Accordingly, raw estradiol data or data from the comparison can also be subject to an algorithm, optionally implemented by a computer or data processor. For example, amounts of estradiol in a sample such as saliva can be determined, and optionally subjected to an algorithm that converts the saliva estradiol amount into a serum equivalent unit (SEU) of estradiol for analysis. Salivary results may then be readily used in context to a greater body of referenced investigations and results.

Examples of algorithms include linear or nonlinear regression algorithms; linear or nonlinear classification algorithm; computational neural network algorithms; support vector machines algorithms; hierarchical analysis or clustering algorithms; hierarchical algorithms using decision trees; kernel based machine algorithms; table look-up algorithms; discriminatory algorithms such as partial least squares algorithms, matching pursuit algorithms, Fisher discriminate analysis algorithms, principal components analysis algorithms, singular value decomposition algorithms; Bayesian probability function algorithms; Markov Blanket algorithms; hidden Markov algorithms; Genetic algorithms; deterministic optimization algorithms; stochastic search optimization or simulated annealing algorithms; recursive feature elimination or entropy-based recursive feature elimination algorithms; algorithms arranged in combination; plurality of algorithms arranged in a committee network; and forward floating search or backward floating search algorithms.

The invention provides kits, which kits include, for example, detection reagents (e.g., an anatibody) packaged into a suitable packaging material. Kit components can be used to detect, measure or analyze estradiol, for example, an antibody that binds to estradiol in saliva to monitor estradiol over a period of time. Accordingly, in one embodiment, a kit includes a detection reagent such as an antibody that allows detection, measurement or analysis of estradiol. In another embodiment, a kit includes competitive immunoassay reagents for the in vitro quantitative measurement of active free estradiol, an estrogenic steroid, in saliva. In one aspect, a kit includes an antibody that binds to estradiol. Estradiol measurements obtained may be used in diagnosis and treatment of various hormonal sexual disorders and can be used to evaluate ovarian function, as set forth herein.

The term “packaging material” refers to a physical structure housing one or more components of the kit. The packaging material can maintain sterility of the components, and can be made of material commonly used for such purposes (e.g., paper, corrugated fiber, glass, plastic, foil, ampules, vials, tubes, etc.). A kit can contain a plurality of components, e.g., two or more detection reagents alone or in combination.

A kit optionally includes a label or insert including a description of the components (type, amounts, etc.), instructions for use in solid phase, in solution, in vitro, in situ, or in vivo, and any other components therein. Labels or inserts can include instructions for practicing any of the methods or uses described herein. For example, instructions for measuring and/or analyzing estradiol amounts to determine peak estradiol, fertility status, egg production status, or status of female for providing eggs for retrieval and subsequent storage, IVF, etc.

Labels or inserts optionally include information identifying manufacturer, lot numbers, manufacturer location and date, expiration dates. Labels or inserts include “printed matter,” e.g., paper or cardboard, or separate or affixed to a component, a kit or packing material (e.g., a box), or attached to an ampule, tube or vial containing a kit component. Labels or inserts can additionally include a computer readable medium, such as a bar-coded printed label, a disk, optical disk such as CD- or DVD-ROM/RAM, DVD, MP3, magnetic tape, or an electrical storage media such as RAM and ROM or hybrids of these such as magnetic/optical storage media, FLASH media or memory type cards.

Kits of the invention can include a single or a plurality of detection reagents. Kits that include a single or a plurality of detection reagents need not have all or a portion of the detection reagents attached or affixed to a support or substrate. In one embodiment, a kit includes a detection reagent (e.g., antibody) that is attached or affixed to a support or substrate. In another embodiment, a kit includes a detection reagent (e.g., antibody) that is not attached or affixed to a support or substrate.

Kits of the invention can further include other materials or reagents useful in assessing levels of estradiol. For example, a kit can also include software or an application or “app.” Additional non-limiting useful materials and substances include, for example, standard (e.g., a sample containing a known quantity of estradiol to which expression results can be compared). Kits can additionally include a computer readable media (comprising, for example, a data analysis program, etc.), control samples, and other reagents for obtaining and/or processing sample and analysis, and analyzing the data so obtained.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described herein.

All publications and patent applications cited in this specification are incorporated herein by reference as if each individual publication or patent application were specifically and individually indicated to be incorporated by reference. The citation of any publication is for its disclosure prior to the filing date and should not be construed as an admission that the invention is not entitled to antedate such publication by virtue of prior invention.

As used in this specification and the appended claims, the use of an indefinite article or the singular forms “a,” “an” and “the” include plural reference unless the context clearly dictates otherwise. In addition, it should be understood that the individual components or combinations of the components described herein, are disclosed by the application to the same extent as if each composition or group of compositions was set forth individually. Thus, selection of a particular component from among a combination of components is within the scope of the invention.

Unless defined otherwise all technical and scientific terms used herein have the same meaning as commonly understood to one of ordinary skill in the art to which this invention belongs. As used herein, “about” means + or −10%. The use of the alternative (e.g., “or”) should be understood to mean one, both, or any combination thereof of the alternatives, i.e., “or” can also refer to “and.”

The terms “comprises,” “includes,” “having” and grammatical variations thereof mean “including but not limited to.” The term “consisting of” means “including and limited to.” The term “consisting essentially of” means that the composition, method or use may include additional ingredients, steps and/or parts, but only if the additional ingredients, steps and/or parts do not materially alter the characteristics of the composition, method or use.

As used in this specification and the appended claims, any numerical range, such as time (duration) range (minutes, hours, days, etc.), dose range, or other integer range is to be understood to include the value of any integer within the recited range and, when appropriate, fractions thereof (such as one tenth and one hundredth of an integer), unless otherwise indicated. For example, although numerical values are often presented in a range format throughout this document, a range format is merely for convenience and brevity and should not be construed as an inflexible limitation on the scope of the invention. Accordingly, the use of a range expressly includes all possible subranges, all individual numerical values within that range, and all numerical values or numerical ranges including integers within such ranges and fractions of the values or the integers within ranges unless the context clearly indicates otherwise. This construction applies regardless of the breadth of the range and in all contexts throughout this patent document. Thus, to illustrate, reference to a range of 5-10, 10-20, 20-30, 30-40, 40-50, 50-75, 75-100, 100-150, and 150-175, includes ranges such as 5-20, 5-30, 5-40, 5-50, 5-75, 5-100, 5-150, 5-171, and 10-30, 10-40, 10-50, 10-75, 10-100, 10-150, 10-175, and 20-40, 20-50, 20-75, 20-100, 20-150, 20-175, and so forth. Further, for example, reference to a series of ranges of 1-60 minutes or hours, 2-48 minutes or hours, 4-24 minutes or hours, 4-18 minutes or hours and 6-12 minutes or hours, includes ranges of 2-60 minutes or hours, 2-24 minutes or hours, 2-18 minutes or hours, 2-16 minutes or hours, etc.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims. The invention is further exemplified by way of the following non-limited examples.

### EXAMPLES

### Example 1

This example describes exemplary materials and methods.

A prospective study design of was employed. 100 subjects were enrolled. Inclusion criteria were patients undergoing controlled ovarian stimulation (COH) for IVF, subjects excluded had undergone 3 prior IVF cycles.

Saliva E2 values were measured and validated using an enzyme immunoassay. Serum E2 values were assessed as routine:

Concurrent serum and saliva E2 values were collected from patients who provided between 3 and 7 samples on different days of their COH IVF cycle. Instructions included no food or drink one hour prior and quick mouth rinse with water just before collection.

Subjects instructed that collection should take 1-5 minutes. Collection at clinic, phlebotomy site or at home.

Salivary estradiol (Sal E2) is a competitive immunoassay. Microtitre plates are coated with rabbit antibodies specific for E2. E2 in standards and unknowns compete with E2 linked to horseradish peroxidase during incubation. Bound horseradish peroxidase is measured by the reaction of the peroxidase enzyme and the substrate TMB (tetramethylbenzidine) creating a blue color. This reaction is stopped with 2M sulfuric acid creating a yellow color. Optical density is read on a standard plate reader at 450 nm, or other suitable wavelength. The amount of estradiol peroxidase detected is inversely proportional to the amount of estradiol present.

The Sal E2 assay was validated for: Inter-assay variability and Intra-assay variability. The coefficient of variation (CV) is the standard deviation of the measurement values divided by the mean of the set. Both inter-assay CV and the intra-assay CV were determined to be <10% indicating accuracy of the test.

### Example 2

This example describes a model for converting salivary estradiol amounts into serum equivalent units (SEU) of estradiol.

Estradiol levels are critical in the monitoring of In Vitro Fertilization (IVF) Cycles for predicting the timing of egg retrieval or collection, and in particular to avoid Ovarian Hyperstimulation Syndrome (OHSS) whereby a patient's ovaries can become very swollen and fluid can leak into the abdominal and chest area. The algorithm was modeled from concurrent E2 samples collected from serum and saliva. The development of an algorithm model to convert saliva estradiol levels to Serum Equivalent Unit (SEU) is exemplified but not limited to the particular algorithm models described herein.

The SEU provides the medical professional with a conversion factor to translate salivary results into a serum equivalent, for which there is a greater database to assess fertility and ovulation. The SEU may be used in computer coding or for the preparation of an APP (application for a smart phone or handheld device).

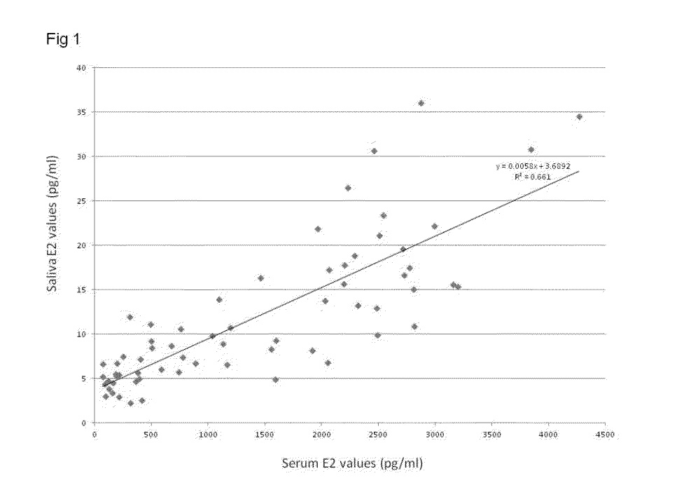

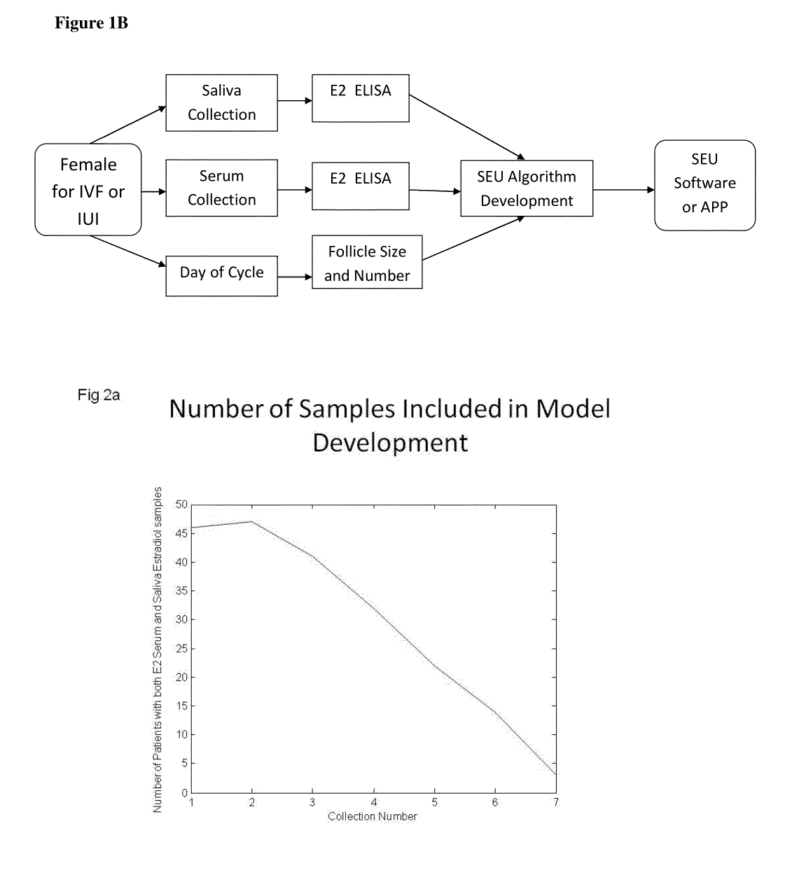

Linear Regression of Serum E2 values and Saliva E2 values of 20 patients who had 3 or more samples evaluated between Days 5 and 14 of their COH cycle. Salivary estradiol measurements were found to have a correlation to those made with serum plasma with a R2=0.661. (FIG. 1).

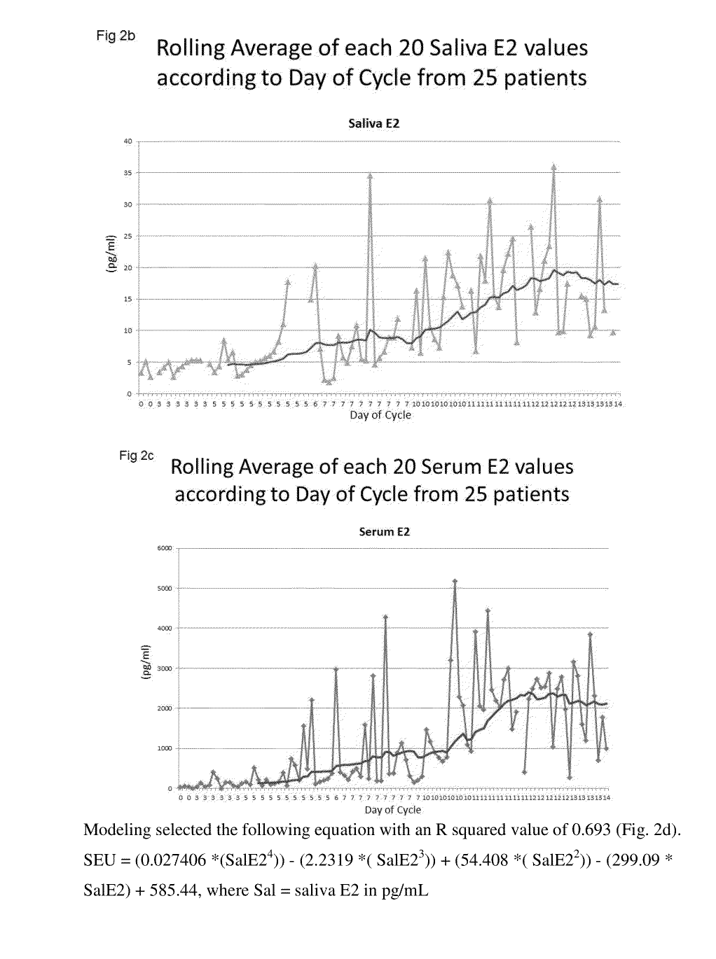

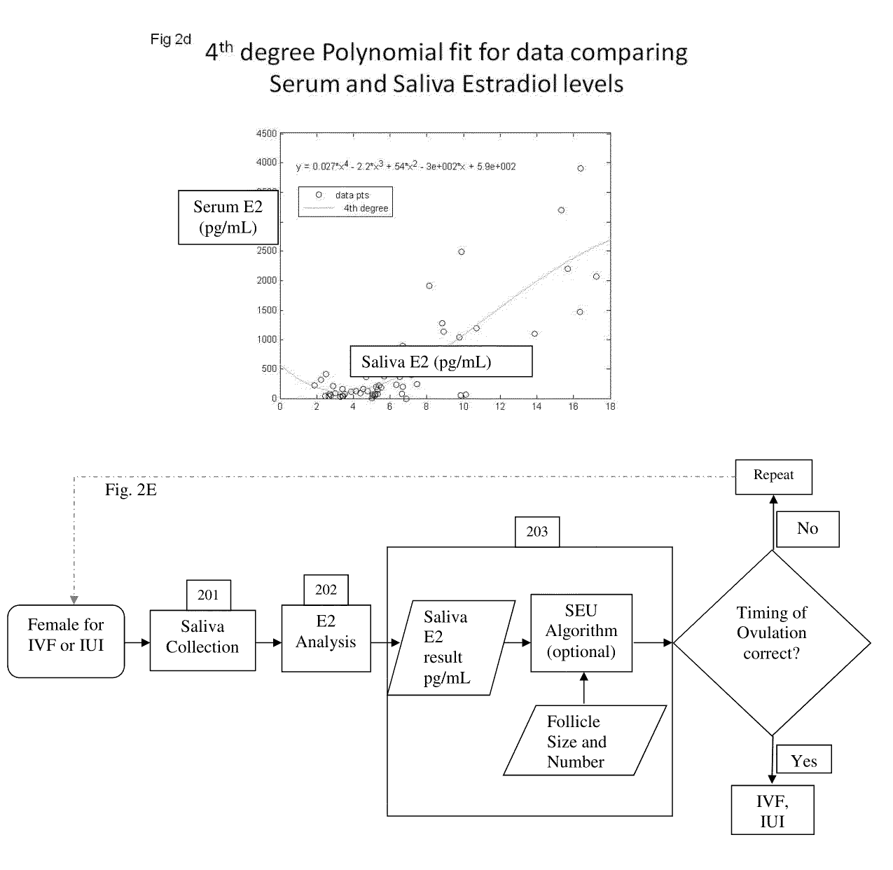

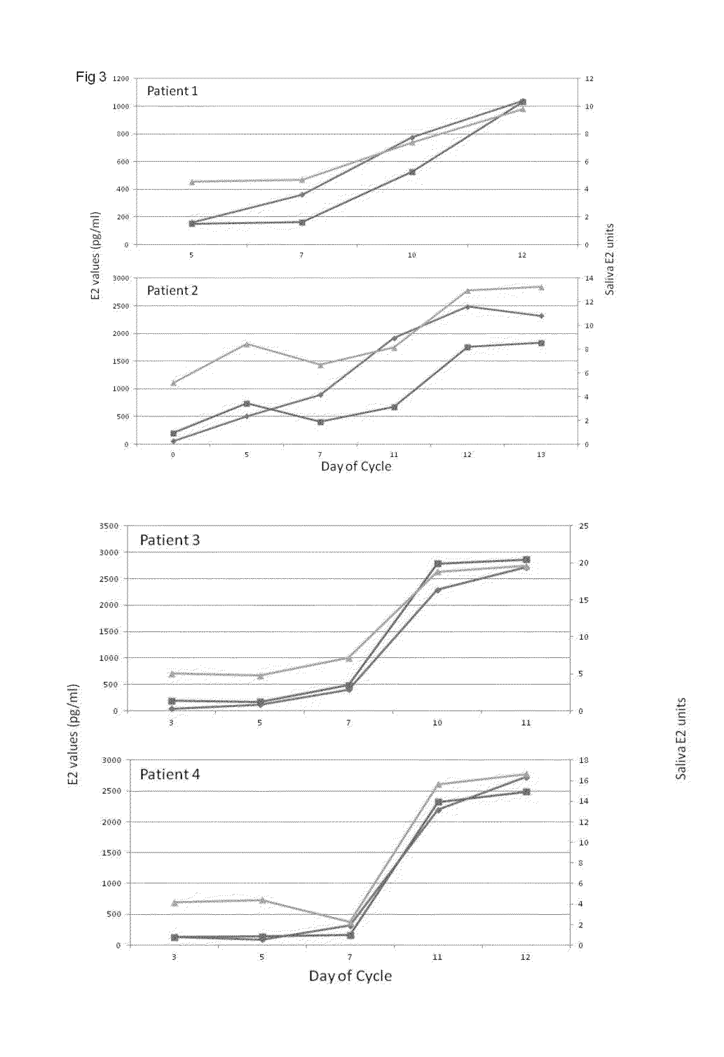

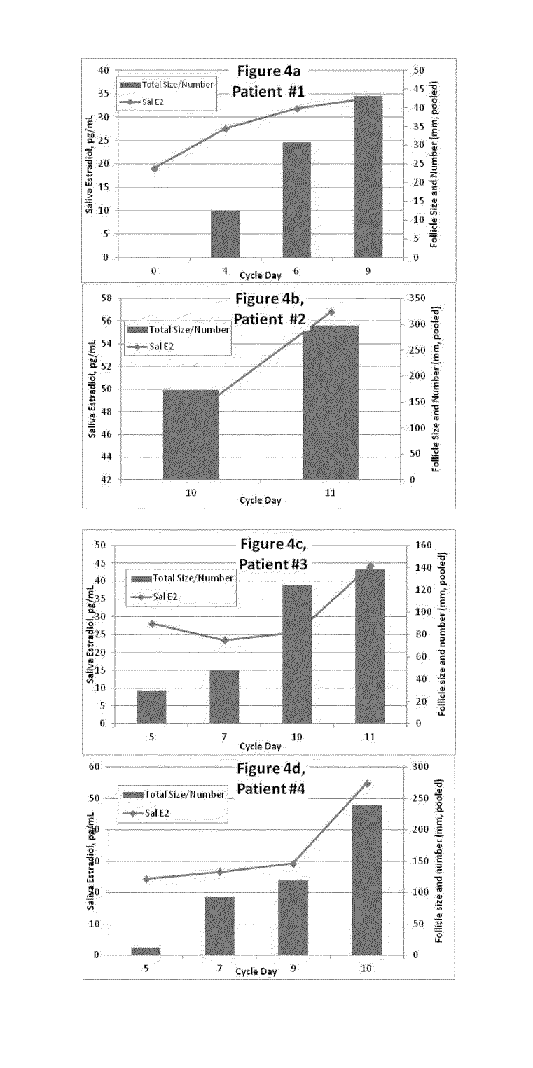

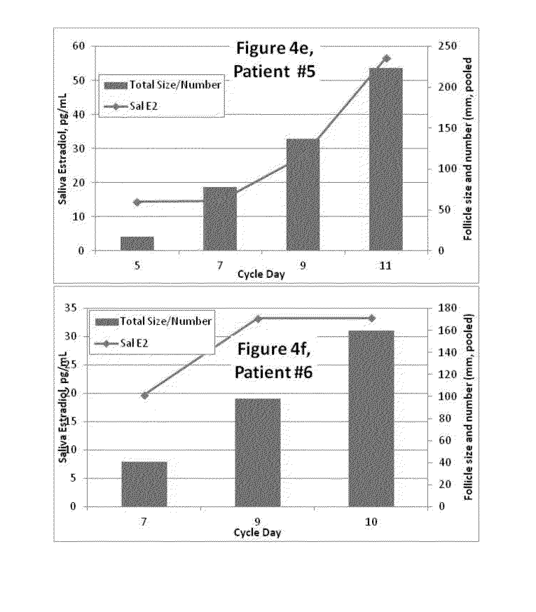

Concurrent Serum and Saliva E2 values were collected from 46 patients who provided between 1 and 7 samples each on different days of their COH Cycle during their In Vitro Fertilization (IVF) treatment (FIGS. 2, 2a, 2b, 2c, 2d, 2e).

Exemplary models were calculated using patient samples with 3 or more collected days of samples. Initial Linear Regression modeling showed a good correlation for comparing Serum and Saliva Estradiol. When separating linear regression models into low (less than 7 pg/ml Saliva Estradiol Levels) and higher values (less than 20 pg/ml Saliva Estradiol Levels) predictability improves at both levels. Given the nature of the non-linear changes in Estradiol levels during the cycle (FIGS. 2c, d) this indicated that the nature of Estradiol levels during the cycle would be better predicted using a polynomial fit. In this case a 4^{th} degree polynomial fit was generated. This would make the use of salivary estradiol more accurate when data is converted into a form representing Serum Equivalent Units. It has also been reported that very high levels of free estradiol stimulates an increase in serum hormone binding globulins which would detract from any direct linear relationship (Ben-Rafael Z, Mastroianni L Jr, Meloni F, Strauss J F 3rd, Flickinger G L. Feral Steril. 1986 October; 46(4):593-8).

The SEU is utilized as depicted in FIG. 2e. The salivary estradiol value is converted to SEU and correlated with follicular size and number to render an opinion and/or decision if the timing of ovulation is correct for IVF and IUI.

The saliva may be collected as passive drool or a swab in a laboratory or by the patient at home (FIG. 2e, 201). The level of estradiol is determined by enzyme immunoassay yielding a result in optical density or luminescence or a lateral flow which could be a point of care device (FIG. 2e, 202). The estradiol level in a digital space, is inputted into a computing device (FIG. 2e, 203) which converts it into SEU and combines the data with follicular size and number. The clinician may then determine if the timing is correct for ovulation.

### Example 3

This example describes monitoring of IVF patients.

Saliva estradiol measurements have been used to monitor in vitro fertilization patients during their cycle and the use of Saliva E2 measurements as an adjunct to the normal follicular size measurements has successfully monitored patients. The estradiol level increases concurrent to the follicular size and number. Ongoing pregnancies have been established using this monitoring technique. Examples of some patients monitored are shown below in Table 1.

| | Cycle | | | |
|---|---|---|---|---|
| Patient | Day | Sal E2 | Left Follicle Sizes | Right Follicle Sizes |
| 1 | 0 | 19.1 | N/A | N/A |
| | 4 | 27.64 | <12 mm (few) | 12.43, 8.8, <12 mm (few) |
| | 6 | 31.91 | 7.37, <12 mm (few) | 13.3, 10.09, <12 mm |
| | | | | (few) |
| | 9 | 34.01 | 11.72, <12 mm (few) | 16.86, 14.57, <12 mm |
| | | | | (few) |
| 2 | 10 | 47.69 | 21.76, 18.77, 17.12, | 19.6, 15.86, 13.72, 13.44, |
| | | | 13.88, 13.5, 12.47, <12 mm | 12.93, <12 mm (few) |
| | | | (few) | |
| | 11 | 56.58 | 19.84, 19.25, 18.74, | 23.76, 20.34, 18.71, |
| | | | 18.3, 17.24, 17.06, | 18.55, 18.02, 15.51, |
| | | | 17.02, 15.5, 13.8, 12.89, | 13.72, <12 mm (few) |
| | | | <12 mm (few) | |
| 3 | 5 | 28.06 | 9.48, <12 mm | 11.14, 9.37, <12 mm |
| | | | (multiple) | (multiple) |
| | 7 | 23.44 | 12.26, <12 mm (one) | 11.9, 11.78, 11.76, <12 mm |
| | | | | (few) |
| | 10 | 25.76 | 17.76, 14.66, 13.75, <12 mm | 18.5, 18.22, 17.24, 13.38, |
| | | | (few) | 10.74, <12 mm (one) |
| | 11 | 44.19 | 21.4, 18.81, 17.92, <12 mm | 21.38, 21.8, 20.18, 16.83, |
| | | | (few) | <12 mm (few) |
| 4 | 5 | 24.27 | 12.79, <12 mm | <12 mm (multiple) |
| | | | (multiple) | |
| | 7 | 26.59 | 15.61, 12.56, 12.1, <12 mm | 13.73, 13.55, 13.12, |
| | | | (multiple) | 12.07, <12 mm (multiple) |
| | 9 | 29.3 | 18.54, 15.26, 14.64, | 14.1, 15.04, 14.76, <12 mm |
| | | | 14.03, 12.81, <12 mm | (multiple) |
| | | | (multiple) | |
| | 10 | 54.8 | 19.48, 18.37, 17.30, | 20.51, 18.50, 18.15, |
| | | | 15.62, 14.85, 12.71, | 15.00, 14.92, 14.44, |
| | | | 12.23, <12 mm (few) | 13.66, 13.59, <12 mm |
| | | | | (few) |
| 5 | 5 | 14.33 | 7.7, <12 mm (multiple) | 9.12, <12 mm (multiple) |
| | 7 | 14.69 | 11.74, 11.46, 10.74, <12 mm | 11.68, 11.46, 11.35, 9.32, |
| | | | (multiple) | <12 mm (multiple) |
| | 9 | 27.79 | 14.92, 14.12, 13.87, | 15.42, 14.48, 14.22, |
| | | | 13.62, 11.11, <12 mm | 13.34, 12.12, <12 mm |
| | | | (few) | (one) |
| | 11 | 56.69 | 20.69, 19.92, 17.58, | 19.94, 18.3, 18.16, 17.86, |
| | | | 16.12, 15.82, 14.4, | 16.69, 14.1, <12 mm |
| | | | 13.96, <12 mm | (multiple) |
| | | | (multiple) | |
| 6 | 7 | 19.69 | 15.48, 14.68 | 10.5 |
| | 9 | 33.28 | 21.88, 17.08, 16.91, | 14.52, 11.32, <12 mm |
| | | | 16.18 | (one) |
| | 10 | 33.31 | 23.05, 22.1922.12, | 15.68, 13.01, 12.13, <12 mm |
| | | | 18.09, 17.35, 16.24 | (one) |

### Example 4

This example describes several non-limiting examples of alternative mathematical algorithms for determining an SEU values from saliva estradiol amounts.

A number of models can be developed when running estradiol analysis on concurrent serum and saliva samples. These can be used to create an algorithm that converts the saliva estradiol values to Serum Equivalent Units (SEU).

An example of some conversion values using different strategies is provided in Table 2, while the ranges of R-squared values calculated using different models is provided in (Table 3). Models can be chosen based on statistical validation showing the best fit to the known Serum Estradiol values when converting Saliva Estradiol values to SEUs.

TABLE 2

| Serum | Salivary | 4th | 3rd | 2nd | |
|---|---|---|---|---|---|
| E2 | E2 | Degree* | Degree** | Degree*** | Exponential**** |
| 92.7 | 4.54 | 152 | 264 | 361 | 252 |
| 109 | 4.23 | 134 | 233 | 286 | 234 |
| 112 | 2.87 | 124 | 200 | −44 | 169 |
| 270 | 3.92 | 121 | 211 | 212 | 218 |
| 338 | 3.61 | 114 | 197 | 137 | 202 |
| 436 | 4.62 | 157 | 273 | 380 | 257 |
| 478 | 5.95 | 296 | 491 | 693 | 354 |
| 1765 | 11.88 | 1515 | 2208 | 2000 | 1459 |
| 1793 | 10.90 | 1286 | 1917 | 1794 | 1154 |
| 1885 | 9.92 | 1059 | 1609 | 1584 | 913 |
| 2324 | 16.62 | 2488 | 2938 | 2938 | 4529 |
| 3501 | 15.89 | 2363 | 2936 | 2800 | 3804 |

*The 4th degree polynomial is shown in FIG. 2d. The following formulas were used to generate the Table where X is the Salivary E2 value
**SEU = −2.5x³ + 73.3x² − 401x + 806
***SEU = −2.1x² + 258x − 766
****SEU = 85.3e^{0.24x}

TABLE 3

| MODEL converting Serum | |
|---|---|
| Estradiol Values to SEU | R-squared ranges |
| Exponential Model | 0.63-0.68 |
| 2nd degree Polynomial | 0.61-0.65 |
| 3rd degree Polynomial | 0.62-0.66 |
| 4th degree Polynomial | 0.65-0.71 |

What is claimed:

1. A method for stimulating production of eggs in a female mammal for in vitro fertilization, comprising,
a.) administering a hormone therapy that stimulates egg production in a female mammal,
b.) obtaining or providing a saliva sample from the female mammal,
c.) measuring the amount of estradiol in the saliva sample,
d.) comparing the amount of saliva estradiol to a previously determined amount of estradiol from the female mammal in order to identify an increase in saliva estradiol, wherein an estradiol peak reflects increased egg production in the female mammal, thereby stimulating egg production in the female mammal for in vitro fertilization.

2. A method for selecting a female for egg production or retrieval, comprising,
a.) administering a hormone therapy that stimulates egg production in a female mammal,
b.) obtaining or providing a saliva sample from the female mammal,
c.) measuring the amount of estradiol in the saliva sample,
d.) comparing the amount of saliva estradiol to a previously determined amount of saliva estradiol from the female mammal in order to identify an increase in saliva estradiol, wherein an estradiol peak reflects increased egg production in the female mammal; and
e.) selecting the female for egg production or retrieval, and optionally retrieving said eggs.

3. A method for providing eggs for in vitro fertilization or increasing probability of pregnancy in a female mammal, comprising,
a.) administering a hormone therapy that stimulates egg production in a female mammal,
b.) obtaining or providing a saliva sample from the female mammal,
c.) measuring the amount of estradiol in the saliva sample,
d.) comparing the amount of saliva estradiol to a previously determined amount of saliva estradiol from the female mammal in order to identify an increase in estradiol, wherein an estradiol peak reflects increased egg production in the female mammal, and
e.) retrieving eggs from the female for subsequent in vitro fertilization, or performing in-utero insemination on the female mammal.

4. The method of any of claims 1-3, comprising repeating steps a.), or b.)-c.), or b.)-d.) a plurality of times in order to determine a peak of estradiol in the female mammal.

5. The method of any of claims 1-4, further comprising determining follicle size or follicle number in the female mammal.

6. The method of any of claims 1-5, wherein the female mammal is selected, the eggs retrieved or the in-utero insemination is performed based upon a combination of any of the amount or peak of saliva estradiol, follicle size and follicle number in the female mammal.

7. The method claim 7, comprising repeating steps a.)-b.) a plurality of times in order to determine amounts of estradiol in the female mammal over a period of time.

8. The method claim 7, comprising repeating steps a.)-c.) a plurality of times in order to determine a peak of estradiol in the female mammal.

9. A method for determining a peak of estradiol in a female mammal, comprising,
a.) obtaining or providing a saliva sample from a female mammal having received a hormone therapy that stimulates egg production,
b.) measuring amounts of estradiol in the saliva; and
c.) repeating steps a.)-b.) a plurality of times in order to determine estradiol in saliva over a period of time and a peak of estradiol in the female mammal.

10. A method for determining increased fertility in a female mammal, comprising,
a.) obtaining or providing a saliva sample from a female mammal, optionally said female having received a hormone therapy that stimulates egg production,
b.) measuring amounts of estradiol in the saliva; and
c.) repeating steps a.)-b.) a plurality of times in order to determine estradiol in saliva over a period of time and a peak of estradiol in the female mammal, wherein the peak of estradiol indicates increased fertility in a female mammal.

11. A method for stimulating production of eggs in a female mammal for in vitro fertilization, comprising,
a.) administering a hormone therapy that stimulates egg production in a female mammal,
b.) obtaining or providing a saliva sample from the female mammal,
c.) measuring amounts of estradiol in the saliva sample,
d.) converting the amount of estradiol measured in the saliva to a serum equivalent unit (SEU) of estradiol;
e.) comparing the SEU of estradiol to a previously determined SEU of estradiol for the female mammal in order to identify an increase in estradiol, wherein an estradiol peak reflects increased egg production in the female mammal, thereby stimulating egg production in the female mammal for in vitro fertilization.

12. A method for selecting a female for egg production or retrieval, comprising,
a.) administering a hormone therapy that stimulates egg production in a female mammal,
b.) obtaining or providing a saliva sample from the female mammal,
c.) measuring amounts of estradiol in the saliva sample,
d.) converting the amount of estradiol measured in the saliva to a serum equivalent unit (SEU) of estradiol;
e.) comparing the SEU of estradiol to a previously determined SEU of estradiol for the female mammal in order to identify an increase in estradiol, wherein an estradiol peak reflects increased egg production in the female mammal; and
f.) selecting the female for egg production or retrieval.

13. A method for providing eggs for in vitro fertilization or increasing probability of pregnancy in a female mammal, comprising,
a.) administering a hormone therapy that stimulates egg production in a female mammal,
b.) obtaining or providing a saliva sample from the female mammal,
c.) measuring amounts of estradiol in the saliva sample,
d.) converting the amount of estradiol measured in the saliva to a serum equivalent unit (SEU) of estradiol.
e.) comparing the SEU of estradiol to a previously determined SEU of estradiol for the female mammal in order to identify an increase in estradiol, wherein an estradiol peak reflects increased egg production in the female mammal, and
f.) retrieving eggs from the female for subsequent in vitro fertilization, or performing in-utero insemination on the female mammal.

14. The method of any of claims 11-13, comprising repeating steps a.) or b.)-d.) a plurality of times in order to determine a peak of estradiol in the female mammal.

15. The method of any of claims 9-14, further comprising determining follicle size or follicle number in the female mammal.

16. The method of any of claims 9-15, wherein the female mammal is selected, the eggs retrieved or the in-utero insemination is performed based upon a combination of any of SEU of estradiol, follicle size and follicle number in the female mammal.

17. A method for determining a serum equivalent unit (SEU) of estradiol in a female mammal, comprising,
a.) obtaining or providing a saliva sample from a female mammal having received a hormone therapy that stimulates egg production,
b.) measuring amounts of estradiol in the saliva sample, and
c.) converting the amount of estradiol measured in the saliva to a SEU of estradiol.

18. The method claim 17, comprising repeating steps a.)-c.) a plurality of times in order to determine amounts of estradiol in the female mammal over a period of time.

19. The method claim 17, comprising repeating steps a.)-c.) a plurality of times in order to determine SEU of estradiol over a period of time and a peak of estradiol in the female mammal.

20. A method for determining a peak of estradiol in a female mammal, comprising,
a.) obtaining or providing a saliva sample from a female mammal having received a hormone therapy that stimulates egg production,
b.) measuring amounts of estradiol in the saliva sample,
c.) converting the amount of estradiol measured in the saliva to a serum equivalent unit (SEU) of estradiol; and
d.) repeating steps a.)-c.) a plurality of times in order to determine SEU of estradiol over a period of time and a peak of estradiol in the female mammal.

21. The method of any of claims 1-20, wherein the estradiol amount or SEU is compared to a baseline level of estradiol or SEU determined in the female mammal prior to the female mammal receiving the hormone therapy that stimulates egg production.

22. The method of any of claims 1-21, wherein a baseline level of estradiol is determined in the female mammal during the estrous cycle prior to the female mammal receiving the hormone therapy that stimulates egg production.

23. The method of any of claims 1-21, wherein the estradiol amount or SEU is compared to a level of estradiol or SEU determined in the female mammal after the female mammal began receiving the hormone therapy that stimulates egg production.

24. The method of any of claims 1-23, wherein the saliva sample is obtained or provided on or from the same day, or within 1-21 days before or after the female mammal began receiving the hormone therapy that stimulates egg production.

25. The method of any of claims 1-24, wherein the saliva sample is obtained or provided on or from the same day, or within 1-14 days before or after the female mammal began receiving the hormone therapy that stimulates egg production.

26. The method of any of claims 1-25, wherein a plurality of saliva samples are obtained or provided on or from before or after the female mammal began receiving the hormone therapy that stimulates egg production, and amounts of estradiol measured in the saliva.

27. The method of any of claims 1-26, wherein a plurality of saliva samples are obtained or provided on or from before or after the female mammal began receiving the hormone therapy that stimulates egg production, and amounts of estradiol measured in the saliva and converted to a serum equivalent unit (SEU) of estradiol.

28. The method of any of claims 1-27, wherein the estradiol in saliva is measured once per day for a period of 1-21 days before or after the female mammal began receiving the hormone therapy that stimulates egg production.

29. The method of any of claims 1-28, wherein the estradiol in saliva is measured once per day for a period of 1-14 days before or after the female mammal began receiving the hormone therapy that stimulates egg production.

30. The method of any of claims 1-29, wherein the estradiol in saliva is measured one or more times 1-30 days before the female mammal began receiving the hormone therapy that stimulates egg production, and one or more times 1-21 after the female mammal began receiving the hormone therapy that stimulates egg production.

31. The method of any of claims 1-30, wherein the estradiol in saliva is measured with a detectable assay.

32. The method of any of claims 1-31, wherein the estradiol in saliva is measured with an antibody immunoassay.

33. The method of any of claims 1-32, wherein the estradiol in saliva is measured by way of an enzyme immunoassay (EIA) or enzyme linked immunosorbent assay (ELISA).

34. The method of any of claims 1-33, wherein the female mammal has received a plurality of hormone administrations.

35. The method of any of claims 1-34, wherein the hormone comprises follicle stimulating hormone (FSH).

36. The method of any of claims 1-35, wherein the peak of estradiol is greater than about 25 pg/mL estradiol in saliva, or wherein the SEU of estradiol is within a range of between about 1,000 and 4,000 pg/mL.

37. The method of any of claims 1-36, wherein hormone therapy is discontinued in the female mammal if saliva estradiol is greater than about 40 pg/mL, or if the SEU of estradiol is greater than about 4,000 pg/mL.

38. The method of any of claims 1-37, further comprising determining follicle size or the number of follicles in the female mammal.

39. The method of any of claims 1-38, wherein where at least 1 or 2 follicles 18 mm or greater in size indicate increased egg production.

40. The method of any of claims 1-39, wherein half or more of all the follicles are at least 15 mm in size.

41. The method of any of claims 1-40, wherein the number of follicles is at least 8.

42. The method of any of claims 1-41, wherein 5-20 eggs are retrieved from the female mammal.

43. The method of any of claims 1-42, further comprising performing in vitro fertilization on one or more of the retrieved eggs, or performing in-utero insemination on the female mammal.

44. The method of any of claims 1-43, wherein the amount of saliva estradiol is converted to the SEU of estradiol using a mathematical algorithm.

45. The method of any of claims 1-44, wherein the amount of saliva estradiol is converted to the SEU of estradiol using a mathematical algorithm set forth as:
SEU=(0.027*(SalE2⁴))−(2.2*(SalE2³))+(54.41*(SalE2²))−(299.1*SalE2)+585.44,
where SalE2 is saliva estradiol amount.

46. The method of any of claims 1-45, wherein the amount of saliva estradiol is converted to the SEU of estradiol using a mathematical algorithm set forth as:
SEU=−2.5x³+73.3x²−401x+806;
SEU=−2.1x²+258x−766; or
SEU=85.3e^{0.24x},
where x=saliva estradiol amount.
